(19) United States
(12) Patent Application Publication
(10) Pub. No.: US 2017/0152076 A1
(43) Pub. Date: Jun. 1, 2017

(54) Portable Medical Venting System

(71) Applicants: Cheryl Louise Sybouts, Spokane, WA (US); Heather Elise Sybouts, Spokane, WA (US)

(72) Inventors: Cheryl Louise Sybouts, Spokane, WA (US); Heather Elise Sybouts, Spokane, WA (US)

(21) Appl. No.: 14/544,225

(22) Filed: Dec. 11, 2014

(51) Int. Cl. B65D 33/01; A61M 39/24; A61M 27/00

(52) U.S. Cl. CPC B65D 33/01; A61M 27/00; A61M 39/24; A61M 2202/02; A61M 2202/0014; A61M 2209/088; A61M 2210/10

(57) ABSTRACT

The present embodiment provides a container for the collection and aspiration of air from a bodily area. Present embodiment comprises an air inlet passage that permits air to flow from a body to a collection chamber in the container and an outlet path for evacuation of air therefrom. Present embodiment is portable, manually operated, and fluid less.

### BACKGROUND

The present utility model embodiment relates generally to the technical field of medical instruments and more specifically it relates to, but is not limited to, the treatment of Prolonged Air Leak¹.

The clinical need for chest drainage arises at completion of pulmonary resection, open cardiac procedures, disease, thoracic trauma and other thoracic procedures. Fluid and air² in the thoracic cavity can interfere with, e.g., organ and tissue function, wound healing, or normal thoracic pressure. Drainage of fluid and air may be performed through the chest wall with a flexible, plastic, chest tube. Said chest tube may be placed percutaneous, and the chest tube passed out through the incision. The location of the chest tube depends on what is being drained, for example, free air in the pleural space rises, so the tube is placed above the second intercostal space at the mid-clavicular line.

Typically once the chest tube is in place it is connected to a chest drainage unit. Typically a rigid container is used for collection, water is poured into the container and a straw which connects to a tube set is placed with its tip below the level of the water to achieve an underwater seal. One type of underwater drainage, which has been used since the 1940's, is a “three-bottle” system which incorporates three chambers to suction and collect blood and other fluids from a patient's thoracic cavity. This system has an underwater seal chamber having a fluid seal which acts as a unidirectional valve for passing fluids and air from the patient's thoracic cavity. Current conventional concept drainage units, known as the “four-bottle system”, add a fourth chamber called a safety seal/manometer chamber, which provides an indicia of the level of suction being applied to the cavity.

The disadvantages of such systems arise after pulmonary resection, open cardiac procedures, thoracic trauma and other thoracic procedures. These are highly invasive procedures wherein Prolonged Air Leak may be the most prevalent postoperative complication. The Journal of Thoracic Disease³ states, “A postoperative air leak is defined by air escaping the lung parenchyma into the plural space after any kind of surgery in the chest”. Some clinicians consider the simple definition of Prolonged Air Leak as continued bubbling of air around a chest tube after being in situ for 48 hours.

Conventional concepts such as cited art, Chest drainage systems and methods EP 2544755 A1 (A chest drainage system includes a collection device and a fluid pathway . . . ), are attached to the distal end of a chest tube for mechanically suctioning air (pneumothorax) or fluid (pleural effusion, blood, chyle), or pus (empyema) from the intrathoracic cavity while the lungs are suction dependent. Initiation of conventional concept cited art is typically connection to a cumbersome water-based pump systems, stationary vacuum source or wall unit and may typically be removed from the patient after being in situ for 48 hours. Although this approach provides immediate post-surgical fluid and air drainage for patients, prolonged use of conventional concepts such as cited art, Chest drainage systems and methods EP 2544755 A1 may be unnecessarily excessive treatment when post-surgical patient's lungs are up and not suction dependent but said surgical patients require additional air drainage due to Prolonged Air Leak.

Similarly, conventional concepts such as sited art, Chest drainage unit U.S. Pat. No. 4,372,336 A ( . . . four-chamber chest drainage system include a collection chamber for collecting blood and other liquids to be drained from the pleural cavity of a patient . . . ) may typically comprise a fluid collection device, a fluid pathway, a water seal container, one or more fluid collection containers and an assembly comprising a motor/pump section. These components also mechanically suction air (pneumothorax) or fluid (pleural effusion, blood, chyle), or pus (empyema) from the intrathoracic space of a patient. However, devices such as Chest drainage unit U.S. Pat. No. 4,372,336A require patients with Prolonged Air Leak to remain in hospital. A European Journal of Cardio-thoracic Surgery⁴ study reported a mean hospital stay of 5 days for patients without Prolonged Air Leak and 10 days for patients with Prolonged Air Leak. Additionally, the incidence of an event, such as, infection, fall, inaccurate or unreliable indications of fluid drainage units and other hazards increase the longer a patient is hospitalized. A study from the Agency for Healthcare Research and Quality⁵ found “. . . length of stay was 22% longer for patients with an event report compared to those without”.

Present embodiment substantially departs from conventional concept such as Chest drainage systems and methods EP 2544755 A1 and Chest drainage unit U.S. Pat. No. 4,372,336 A. The present embodiment's objective is to improve upon conventional concept by providing a fluid-less, light weight, and portable air venting system that allows patients shorter hospital stays and hence fewer events.

By transitioning from conventional concepts fluid suction drainage to present embodiment the patient is able to mobilize in the hospital or be discharged to the comfort of home with active venting of air.

Early conventional concepts utilized a glass bottle and glass tube immersed underwater creating an atmospheric vacuum to suction fluid into a collection bottle. Newer conventional concepts generally are either a wall mounted central vacuum line or cumbersome mechanical vacuum pump connected to an electrical outlet. For example conventional concepts such as sited art, Chest drainage unit U.S. Pat. No. 4,747,844 A ( . . . chest drainage apparatus characterized by a bottle housing fluid collection chamber and fluid inlet . . . ) typically comprise the bottle housing, the fluid collection chambers, and the fluid inlet, along with an exhaust port, negative pressure relief valves, reservoirs of chest fluids and an assembly comprising a motor/pump section.

Designed primarily for in-patient use, conventional concepts are typically large, bulky pump systems which decrease or altogether eliminate portability and complicate transportation or ambulation effectively immobilizing postoperative patients until suction can be removed.

Mobility management for a patient transitioning off of conventional concepts such as sited art, Chest drainage unit U.S. Pat. No. 4,747,844 A to the present embodiment, a smaller, more mobile device, is key to patient recovery. Early ambulation can help restore normal pulmonary function, enhance circulation, create a more positive patient mood through increased independence, and the like.

Present embodiment may allow the medically stable, post-surgical patient whose lungs are up and not suction dependent, but said post-surgical patient with active draining air from sites proximate to surgical procedures, to mobilize in a hospital or to be discharged to home.

Conventional concepts of some cited art, such as Container WO 2009027665 A1 (A drainage bag is mainly used in ambulatory applications, emergencies, ambulances or military applications or where patients are transported, as drainage bags are more compact, lightweight and flexible than bottles . . . ) are designed to evacuate fluid and air when the lungs can be subject to collapse or incomplete expansion. Conventional concepts of some cited art, such as Container WO 2009027665 A1 applications are fluid chest drains for emergencies, ambulances or military applications or where patients are transported. With conventional concepts of some cited art, such as Container WO 2009027665 A1 the venting air is compressed by the fluid entering the bag. The purpose of venting air is to allow additional fluid to enter.

Such drainage bags have an additional disadvantage which arises from the fact that it may not always be possible to suspend the bag vertically. If the bag is resting on the patient's bed or stretcher, a part of the bag may be folded or otherwise obstructed inhibiting entry of fluid to, or escape from, the bag.

Present embodiment is selectively designed for air and not fluid. The present embodiment substantially departs from conventional concepts, such as cited art Container WO 2009027665 A1 as present embodiment may be deployed when the post-surgical patient's lungs are up and not suction dependent but said post-surgical patient requires additional air drainage. Present embodiment may be selective for air venting and thus does not have bodily fluids to manage.

An additional advantage with present embodiment maybe the simple structure which allows convenience of usage, handling and deployment. Such simplicity significantly reduces any potential interference that would inhibit effective air venting. Present embodiment may be operational whatever the orientation of the container.

Conventional concept such as cited art, Portable suction apparatus WO 1999010024 A1 ( . . . a portable device for the collection of thoracic fluid comprising a collection chamber. The collection chamber and suction unit are of a size and weight that allow them to be carried by a patient . . . ), are designed to collect thoracic fluid restricting conventional concepts useful application to the elimination of fluid buildup in the thoracic cavity. Consequently conventional concepts are incapable of dealing prudently with the practical matters of treating conditions such as Prolonged Air Leak. Though conventional concept is portable, it is still a mechanically based unit designed for fluid and air drainage. Conventional concept such as cited art Portable suction apparatus WO 1999010024 A1 have disadvantages associated with fluid drainage units such as disruption of the suction mechanism, liquid waste-fluid bag disposal and power source monitoring. Present embodiment is simple in structure providing manual aspiration for the dry venting release of collected air to the atmosphere.

Conventional concepts such as cited art, Chest drainage apparatus with check valve U.S. Pat. No. 4,738,671 A (This invention relates to . . . improved chest drainage apparatus characterized by a bottle housing a fluid collection chamber and a fluid inlet connectible to receive fluid and air from a chest cavity . . . a U-tube between the fluid inlet and the air exhaust port . . . a subassembly including main air chamber containing a check valve . . . and a negative pressure relief valve connected to the air intake port . . . subassembly is detachable from the bottle and the latter is used by itself in inverted position as a reservoir of chest fluids that may be reintroduced back into the body . . . positive pressure relief valve downstream of the check valve . . . ), are complex systems that involve a large number of parts that may pose a higher risk of being knocked over or that may be damaged during transportation of patients with adverse consequences.

Conventional concepts such as cited art, Chest drainage apparatus with check valve U.S. Pat. No. 4,738,671 A, require continuous monitoring of gages, flow meters, internal drains and suction lines for clogs, water column and vacuum levels, and the like. Additional disadvantage of conventional concepts such as cited art, Chest drainage apparatus with check valve U.S. Pat. No. 4,738,671 A, are the difficulties associated with cleaning and sterilization of conventional concept for re-use. These disadvantages ultimately increase the time clinicians must devote to the device. Conventional concepts such as cited art, Chest drainage apparatus with check valve U.S. Pat. No. 4,738,671 A, are complex, labor-intensive machines that need to be maintained and sanitized for a patient's benefit and comfort. Such complexities add increased failure potential and undesirable expense to the treatment without achieving substantial gains in patient outcome. Present embodiment is substantially more efficient. Present embodiment may be simple in structure and convenience of operation as it requires little in the way of clinician training or precision, and it provides easy, near instant assembly. Additionally, present embodiment may be completely disposable, and may be discarded after use eliminating the need for cleaning and sanitizing of any equipment involved. Further, conventional concepts such as cited art, Chest drainage apparatus with check valve U.S. Pat. No. 4,738,671 A, confine patients primarily to their beds requiring patients eligible for discharge mandated additional days in the hospital. Again referencing the European Journal of Cardio-thoracic Surgery⁴ study which reported a mean hospital stay of 5 days for patients without Prolonged Air Leak and 10 days for patients with Prolonged Air Leak. With conventional concepts the treatment of patients with conditions such as Prolonged Air Leaks can persist for days or even weeks with patients tethered to conventional concepts. These shortcomings dramatically increase length of hospital stay and markedly increase medical expenses for patient and hospital.

The shortcomings of the numerous cited conventional concept drainage systems afore mentioned can be overcome by the substantial and significant new features of present embodiment. Present embodiment can produce valuable, unexpected new results. Present embodiment may be fluid less, being selectively designed for air and not liquid. Present embodiment may be simple in structure and may be convenient to operate.

Present embodiment requires little in the way of precision as it provides a near instant assembly. Present embodiment may be easily deployed by a patient allowing ambulation in the hospital or in their homes. Present embodiment may dramatically shorten hospital stays providing a cost savings to patients and hospitals.

### BRIEF DESCRIPTION OF DRAWINGS

FIG. 1: diagrammatic view showing approximate placement of the venting bag (106) in relationship to the body and wound (88) with view showing venting bag (106) mounted approximately on patient's thigh.

FIG. 2:
a. diagrammatic view showing an adapted outlet nozzle (28) at the unidirectional valve distal end (26) and air inlet passage proximal end (32) communication in disconnect mode.
b. exploded diagrammatic view showing the adapted outlet nozzle (28) at the unidirectional valve distal end (26) and air passage proximal end (32) communication in disconnect mode.
c. diagrammatic view showing the air inlet passage (34) cut to size at the air inlet passage proximal end (32).

FIG. 3:
a. diagrammatic view showing the unidirectional valve distal end (26) and air inlet passage proximal end (32) in connected mode with securing grip⁷ (30).
b. exploded view showing securing grip (30) in the connected mode imbricating the unidirectional valve distal end (26) and air inlet passage proximal end (32).

FIG. 4: In an alternate embodiment a channel valve (108) is configured into the container and being embedded, welded, fused, glued, adhered, bonded, sealed, or otherwise attached.

FIG. 5: diagrammatic view of the venting bag (106).

FIG. 6:
a. diagrammatic view of the vent assembly base (86) in deployment exposing deflation slit (92).
b. diagrammatic view showing evacuation of captured PAL air (84) from the venting bag (106).
c. cross sectional view of the vent assembly base (86).

### DETAILED DESCRIPTION OF DRAWINGS

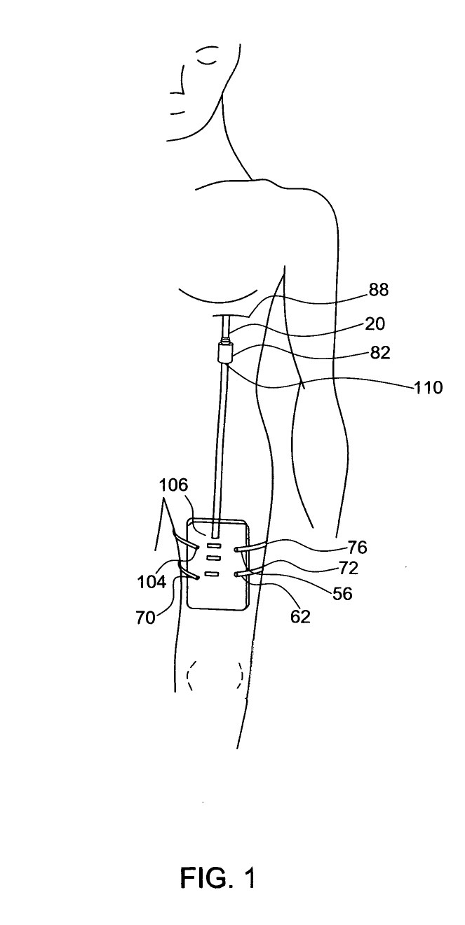

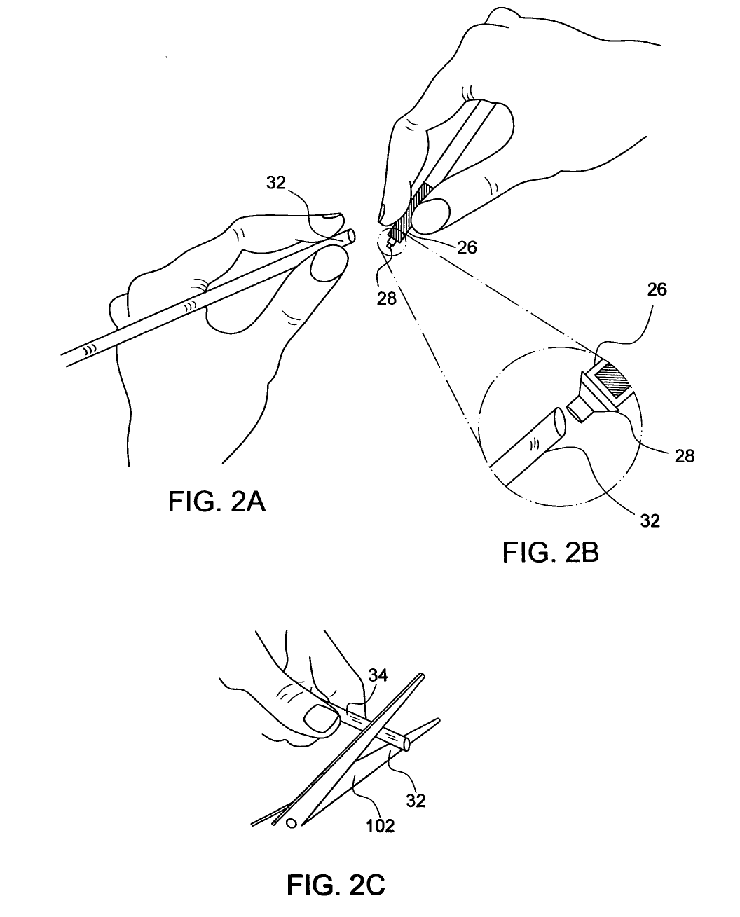

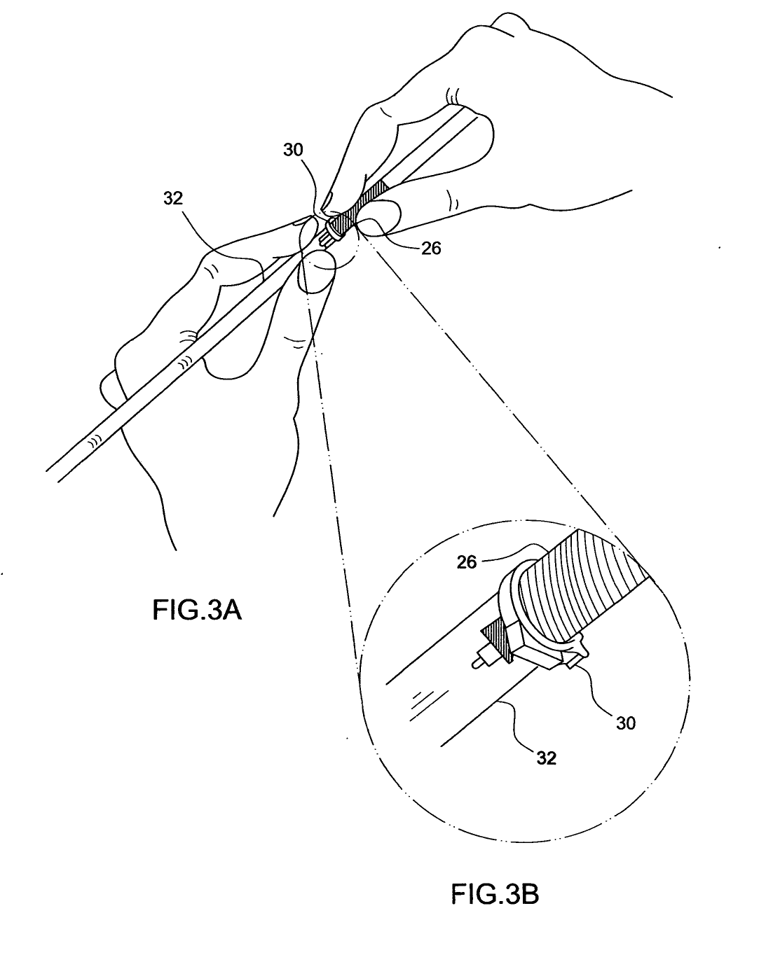

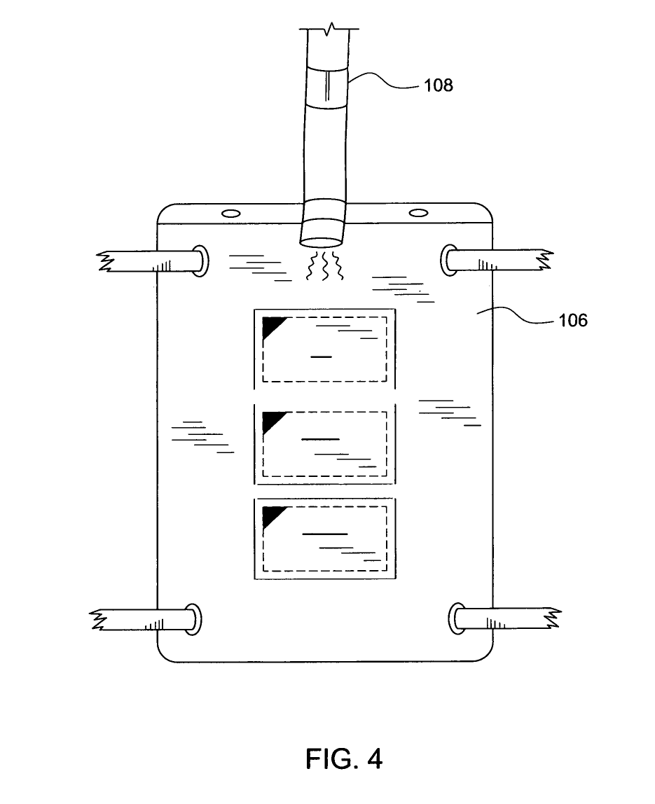

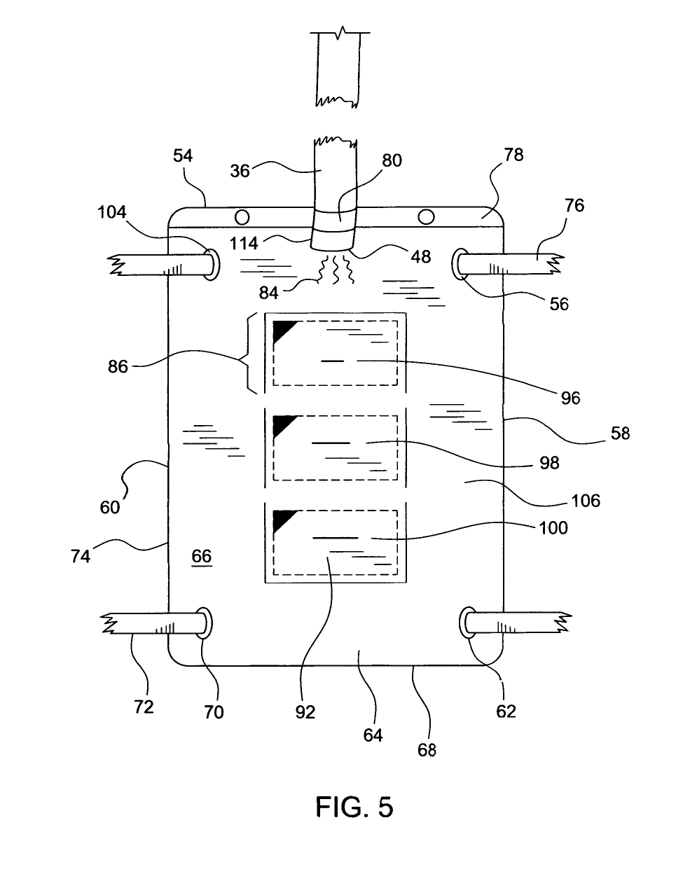

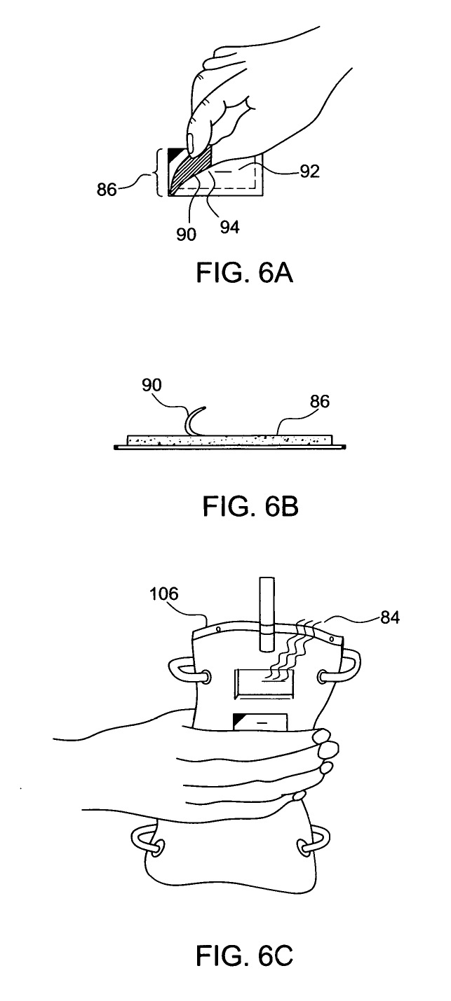

FIG. 1: diagrammatic view with approximate placement of venting bag (106) in relationship to the body and wound (88). The optimal location of chest tube (20) is dependent upon clinical need at completion of pulmonary resection, open cardiac procedures, disease, thoracic trauma and other thoracic procedures⁸. Diagrammatic view, shows the wound (88), an established in practice unidirectional valve (82), an established in practice unidirectional valve-air inlet passage proximal end connection (110) and the venting bag (106) mounted approximately on patient thigh. The mounting member comprises a right top strap aperture (104), a left top strap aperture (56), a right bottom strap aperture (70), a left bottom strap aperture (62), a top mounting strap (76) and a bottom mounting strap (72). The top mounting strap (76) and bottom mounting strap (72) may be detachable and adjustable leg-encircling suspension straps, comprising a releasable fastening means, such as, but not limited to, an adhesive, a clip, a hook and eye, a button, a zip, Velcro™, a combination thereof or the like thereby providing reliable attachment, good mobility and convenience of use.

FIG. 2:
a. diagrammatic view showing an adapted outlet nozzle (28) at the unidirectional valve distal end (26) and air inlet passage proximal end (32) communication in disconnect mode.
b. exploded diagrammatic view showing the adapted outlet nozzle (28) at the unidirectional valve distal end (26) and air inlet passage proximal end (32) communication in disconnect mode.
c. diagrammatic view showing the air inlet passage (34) wherein the overall length may be comprised such as but not limited to the length required to sufficiently accommodate motion or patient movements and anatomical variations. View shows adjusting the length at the air inlet passage proximal end (32) with scissors (102).

FIG. 3:
a. diagrammatic view showing unidirectional valve distal end (26) and air inlet passage proximal end (32) in connected mode with securing grip (30).
b. exploded view showing securing grip (30) in connected mode imbricating the unidirectional valve distal end (26) and air inlet passage proximal end (32) connection. This connection should be adequately strong, reliable and tight for securing the preferred embodiment when in use to prevent preferred embodiment from becoming disturbed, damage or entangled with other equipment. Further, the connection should preferably be releasable, making it possible both to disconnect and connect. Still further, the connection operation should preferably be simple, quick and reliable.

FIG. 4: diagrammatic view of an alternate embodiment channel valve (108) configured into the container (106). Channel valve (108) may be embedded, welded, fused, glued, adhered, bonded, sealed, or otherwise attached.

FIG. 5: a diagrammatic view of the venting bag (106). The body of the container may be a venting bag (106) comprising a pair of opposed walls, front side (64) and back side (60) and may be comprised of an upper delimiting edge (54), a lower delimiting edge (68), a right lateral delimiting edge (74) and a left lateral delimiting edge (58). The venting bag (106) materials may be welded, fused, glued, adhered, bonded, sealed, embedded or otherwise attached together along at least a substantial portion of their peripheral edge delimiting a closed interior space, the collection chamber (66). The container may be of a size and weight that allows ease of mobility. The upper delimiting edge (54) of the venting bag (106) may comprise a reinforcing material formed from semi-rigid or rigid material, fused, glued, adhered, bonded, embedded or otherwise attached to the venting bag (106) material to reinforce and provide strength to an inlet path support member (78). The upper delimiting edge (54) may comprise a centered stoma (80) configured to receive the air inlet passage distal end (36). The stoma (80) may comprise at least an outer surface and an inner surface, and a proximal end and a distal end. The proximal end of the stoma (80) may receive and enclose, without oblation, the air inlet passage distal end (36). The overall length of the air inlet passage distal end (36) may be comprised such as but not limited to approximately the length required to sufficiently extend from the outer surface of the upper delimiting edge (54) to a collection chamber (66) of the venting bag (106). The length of the stoma (80) between the proximal end and distal end preferably is not greater than the length of the air inlet passage distal end (36) such that a terminus air inlet passage (114) protrudes through the stoma (80), into the venting bag (106) collection chamber (66). The air inlet passage terminus (114) comprises a stoma valve opening (48) through which the captured PAL air (84) is vented into the collection chamber (66). The front side (64) of the venting bag (106) comprises a vent assembly base (86) comprising a reinforcing semi-rigid or rigid material. This connection is fused, glued, adhered, bonded, embedded or otherwise attached. The vent assembly base (86) comprises at least one deflation slit (92) aperture extending from approximately the outer surface to inner surface of the venting bag (106)-vent assembly base (86) communication. The vent assembly base (86) may be comprised of one or a plurality of deflation slit (92) apertures. The deflation slit (92) apertures preferably descend in size from one end to an opposite end of the vent assembly base (86). For example; the vent assembly base (86) may be comprised of varying deflation slit (92) apertures being, but not limited to, slit interstitial 0.5 cm (96), slit interstitial 1.5 cm (98), slit interstitial 2.0 cm (100) respectively. One or more deflation slits (92), may be exposed during expulsion of air. The mounting member may comprise an attachment means comprising a right top strap aperture (104), a left top strap aperture (56), a right bottom strap aperture (70), a left bottom strap aperture (62), a top mounting strap (76) and a bottom mounting strap (72). The top mounting strap (76) and bottom mounting strap (72) may be detachable, adjustable leg-encircling suspension straps, comprising a flexible material, releasable fastening means, such as, but not limited to, an adhesive, a clip, a hook and eye, a button, a zip, Velcro™, a combination thereof or the like thereby providing reliable attachment, good mobility and convenience of use. These mounting straps may be secured to the patient's thigh or other bodily area and adjusted for proper fit and comfort.

FIG. 6:
a. diagrammatic view showing the vent base assembly (86). The vent base assembly (86) comprises a hold-out area (94) formed in a peel off adhesive vent cover (90). The hold-out area (94) may be arranged to permit at least a portion of the vent cover (90) to be grasped by a user. The view shows deployment by exposing a deflation slit (92) by grasping and pulling the hold-out area (94) thereby substantially removing the peel off adhesive vent cover (90).
b. diagrammatic view comprising the vent cover (90) mounted externally, wherein the vent cover (90) and the vent base assemble (86) are substantially the same size with the vent cover (90) substantially superimposed over the vent base assemble (86). The inner side of the vent cover (90) may be secured to the outer side of the vent base assemble (86).
c. diagrammatic view showing manual activation by compression, aspirating the venting bag (106) for selectively controlling and evacuating of captured PAL air (84).

### FIGURE NUMBERS

20. Chest tube

26. Unidirectional valve distal end

28. Adapted outlet nozzle

30. Securing grip

32. Air inlet passage proximal end

34. Air inlet passage

36. Air inlet passage distal end

38. Mesial air inlet passage

48. Stoma valve opening

52. Suspension apertures

54. Upper delimiting edge

56. Left top strap aperture

58. Left lateral delimiting edge

60. Back side

62. Left bottom strap aperture

64. Front side

66. Collection chamber

68. Lower delimiting edge

70. Right bottom strap aperture

72. Bottom mounting straps

74. Right lateral delimiting edge

76. Top mounting straps

78. Inlet path support member

80. Stoma

82. Established in practice unidirectional valve

84. Captured PAL air

86. Vent assembly base

88. Wound

90. Vent cover

92. Deflation slit

94. Hold out area

96. Slit Interstitial 0.5 cm

98. Slit Interstitial 1.5 cm

100. Slit Interstitial 2.0 cm

102. Scissors

104. Right top strap aperture

106. Venting Bag

108. Channel valve

110. Established in practice unidirectional valve-Air inlet passage proximal end connection

114. Air inlet passage terminus

116. Channel valve distal end

118. Channel valve proximal end

### SUMMARY

According to present embodiment there is provided a container suitable for connection to an established in practice unidirectional valve for the collection and evacuation of air from, for example, a thoracic cavity. Present embodiment container may comprise an air inlet passage, a venting bag, a vent base assembly, and an adjustable leg-encircling mounting member. The container may be a medical/surgical container or bag. The present embodiment container may be sized and shaped to allow use as an ambulatory container or bag.

At completion of pulmonary resection, open cardiac procedures, thoracic trauma and other thoracic procedures, a flexible, plastic, chest tube may be placed percutaneous, and said chest tube passed out through the incision. The location of the chest tube is dependent on the clinical need at completion of pulmonary resection, open cardiac procedures, disease, thoracic trauma and other thoracic procedures. The chest tube distal end extends from the thoracic space typically attaching to the proximal end of an established in practice unidirectional valve. The unidirectional valve allows forward flow and prevents backflow of air. The present embodiment may attach to the established in practice unidirectional valve distal end. To engage the present embodiment the air inlet passage proximal end is preferably adjusted to size to an overall length such as but not limited to the length required to sufficiently accommodate motion, patient movement, anatomical variation, and such. An easy connection may then be made between the established in practice unidirectional valve and the present embodiment air inlet passage proximal end. The established in practice unidirectional valve may comprise a distal end with an adapted outlet nozzle configured to receive the air inlet passage proximal end. This established in practice unidirectional valve-air inlet passage proximal end connection may comprise at least one removable attachment such as a securing grip by which the members are held together. Said connection should be adequately strong, reliable and tight for securing present embodiment when in use to prevent present embodiment from becoming disturbed, damaged or entangled with the patient, other equipment, and such. Further, the connection should preferably be releasable, making it possible both to disconnect and reconnect. Still further, the connection operation should preferably be simple, quick and reliable.

The air inlet passage may be comprised of a hollow body simple in structure, commonly made from flexible and resilient material, such as but not limited to plastic or polymer. The air inlet passage may provide an overall air path from the unidirectional valve to the venting bag.

The venting bag comprises a pair of opposed walls, front side and back side and may be comprised of an upper delimiting edge, a lower delimiting edge, a right lateral delimiting edge and left lateral delimiting edge with said edges delimiting a space, an interior collection chamber. The venting bag materials may be welded, fused, glued, adhered, bonded, sealed, embedded or otherwise attached together along at least a substantial portion of their peripheral edge. The venting bag is preferably comprised of a soft, resilient, flexible membrane such as but not limited to plastic or polymer; however other materials may be utilized in the construction. Such materials may increase comfort during treatment when the venting bag membrane may be placed next to the patient's skin. Such materials may be convenient to wear as the bag membrane may reduce potential interference with clothing. The size, shape and weight of the container may be such that ambulatory use, easy mounting, reliable attachment, good mobility and convenience of use are feasible.

The mounting member may comprise an attachment means of a flexible material comprising a right top strap aperture, a left top strap aperture, a right bottom strap aperture, a left bottom strap aperture, a top mounting strap and a bottom mounting strap. The top mounting strap and bottom mounting strap may comprise a releasable fastening means, such as, but not limited to, an adhesive, a clip, a hook and eye, a button, a zip, Velcro™, a combination thereof or the like. These suspension straps may be secured in a fixed relationship and arranged to be mounted on a patient to sufficiently accommodate motion, patient movement, anatomical variation, comfort and such.

The upper delimiting edge of the venting bag may comprise at least one reinforcing member, the inlet support member, comprising reinforcing material formed from semi-rigid or rigid material, fused, glued, adhered, bonded, embedded or otherwise attached to the venting bag material to reinforce and provide strength to the inlet path support member. The upper delimiting edge of the venting bag may comprise a centered stoma configured to receive the air inlet passage distal end. Said stoma comprises at least an outer surface and an inner surface, and a proximal end and a distal end. The proximal end of the stoma may receive and enclose, without oblation, the air inlet passage distal end.

The overall length of the air inlet passage distal end may be comprised such as but not limited to the length required to sufficiently extend from approximately the outer surface of the upper delimiting edge of the venting bag to the interior collection chamber of the venting bag. The length of the stoma between the proximal end and distal end preferably is not greater than the length of the air inlet passage distal end such that, in use, the of the air inlet passage terminus protrudes through the stoma, into the venting bag collection chamber. The air inlet passage terminus comprises a stoma valve opening through which the captured PAL air is vented into a collection chamber.

A means is provided for venting the bag, under controlled circumstances, to exhaust captured PAL air into the atmosphere. In present embodiment the venting bag may comprise at least one vent assembly base arranged communally with the venting bag comprising a reinforcing material formed from semi-rigid or rigid material such that bending or folding is inhibited. Such reinforcing material, may be fused, glued, adhered, bonded, embedded or otherwise attached. A peel off adhesive vent cover may be substantially superimposed over the vent base assembly. The vent base assembly and the vent cover preferably are substantially the same size. An adhesive-free hold-out area may be formed in the vent cover. The hold-out area may be arranged to permit at least a portion of the peel off adhesive vent cover to be grasped by a user. A configuration to permit repeated unsealing and resealing of the vent cover may be provided.

The vent assembly base may comprise of one or more deflation slit apertures extending preferably from the outer surface to inner surface of the venting bag-vent assembly base for control of and expulsion of air from the container. The deflation slits may be located in the most suitable configuration for the particular application. For example, the deflation slit apertures may preferably descend in size from one end to an opposite end of the vent assembly base. The vent assembly base may be comprised of one or a plurality of deflation slit apertures, for example, but not limited to, slit interstitial 0.5 cm, slit interstitial 1.5 cm, slit interstitial 2.0 cm respectively. The peel off adhesive vent cover may be configured so as to allow the exposure of one or more deflation slits during the expulsion of air from the container.

The captured PAL air, having entered the bag, may be maintained in the collection chamber until manually aspirated. A medical clinician may select one or more deflation slit apertures according to patient's needs. The selected deflation slit aperture-peel off adhesive vent cover may be skived back. The user may then gently cup hands around the venting bag and preferably, with relatively small reaction force, squeeze the venting bag aspirating captured PAL air to atmosphere.

Thus, when in use, preferred embodiment provides an easy mechanism through which air moves from a thoracic cavity, through the air inlet passage, and into the venting bag collection chamber whereby it may be expelled to atmosphere.

It is recognized that various alterations and modifications may be required due to patient needs, hospital supplies, clinician preferences and such. To provide for these and other unforeseen needs, the embodiment may have additional advantages. For example, in the event a unidirectional valve is not established in practice, an alternate embodiment may comprise a one-way valve which may be configured, embedded, welded, fused, glued, adhered, bonded, sealed, or otherwise attached to the container. A one-way valve may allow a forward flow and prevents backflow of air.

### CONCLUSION

The present embodiment may provide a substantially and significantly unique embodiment for the collection and aspiration of air from a bodily cavity. Present embodiment comprises an air inlet passage that permits air to flow from a body cavity to a collection chamber in the container and an outlet path for evacuation of air therefrom. The present embodiment offers new and unexpected advantages. A number of these important and significant advantages may have become evident:
1. Present embodiment is fluid less, being selectively designed for air and not liquid.
2. Present embodiment, being selectively designed for air, does not have bodily fluids to manage and no liquid waste to dispose.
3. Present embodiment being simple in structure allows convenience of usage, handling and deployment. Present embodiment may be substantially more efficient than conventional concepts as present embodiment may be manual, not mechanical.
4. Present embodiment requires little in the way of training or precision as present embodiment may provide a near instant assembly for the clinician.
5. Present embodiment may allow the medically stable patient, with active draining air from sites proximate to surgical procedures to easily deploy present embodiment allowing ambulation in a hospital or home.
6. Present embodiment may dramatically shorten hospital stays in turn providing a cost savings to hospitals and patients. Shorter hospital stays reduce the incidence of an event, such as, infection, fall, inaccurate or unreliable indications of fluid drainage units and other hazards may be reduced.
7. Present embodiment may allow earlier ambulation which may restore normal pulmonary function, create a more positive patient mood through increased independence, enhance circulation, and the like.
8. Present embodiment may be completely disposable, and may be discarded after use eliminating the need for cleaning and sanitizing of equipment involved.

### Ramifications, Description and Operation of Alternative Embodiments

Although the embodiment of the invention has been described using specific terms, such description should not be construed as a limitation on the invention and it is to be understood that changes and variations may be made without departing from the spirit or scope of the invention.

Other ramifications and variations are possible within the teachings of the embodiments. It is recognized that various alterations, dimensions, alternate usages, material, shape, size, method of operation, embodiment, modification, and such may be required due to patient need, hospital supplies, environments, clinician preference and such. It is also recognized that elements may be eliminated or duplicated, inter connected, connected or associated with adjacent elements in a different manner, made integrally or separately, or be given a different mode or function of operation. To provide for these and other unforeseen need the embodiment may have, but is not limited to, additional advantages. For example;
1. In an event where a unidirectional valve is not established in practice, alternate embodiment may comprise a channel valve which may be configured, embedded, welded, fused, glued, adhered, bonded, sealed, or otherwise attached to the container. A channel valve may allow a forward flow and prevents backflow of air. Further, a connection should preferably be made releasable, making it possible both to disconnect and reconnect. Still further, the connection operation should preferably be simple, quick and reliable. The valve may be configured, embedded, welded, fused, glued, adhered, bonded, sealed, or otherwise attached to the container.
2. Present embodiment may be suspended at bedside. Two suspension apertures (52, FIG. 5) are provided at the upper delimiting edge (54, FIG. 5) of the container for such purpose. Further present embodiment may be carried, attached to wheelchair, patient's clothing or such.
3. Present embodiment may comprise a reduction coupler⁶ to engage instruments of variant sizes and dimensions. A reduction coupler comprising a coupling body having a hollow passage with an enlarged entrance which reduces to a smaller entrance at opposite ends thereof. The reduction coupler may be employed, for example, when materials of variant size are employed.
4. Present embodiment relates generally to the technical field of medical instruments. Additions and alterations may be introduced into the construction and arrangement of the embodiment without departing from the scope of the present invention. I.e. the present embodiment may be adapted for any market, such as but not limited to, veterinary medicine.

### Glossary

air: generally air in or around the thorax, air is also known as gas, pneumothorax and the like
air inlet passage: a hallow tube used as a conduit to drain air from a body cavity
ambulation: walking about or able to walk about; one who is not confined to bed or hospital
aspiration: removal of air from i.e.: a body cavity such as a thoracic cavity or venting bag
aperture: an opening, as a hole
channel valve: a unidirectional valve embedded or otherwise attached to the container
chest tube: tube inserted through the rib space of the thorax into the pleural space to remove air and/or fluid, a drainage path from the body
chest drain: a device that suctions fluid, air and pus from a thoracic cavity
chyle: a turbid white or pale yellow fluid
collection chamber: the interior air bladder or sack of the venting bag
coupler: a small, hallow plastic device that links things together
deflation slit: a narrow, lengthwise, straight incision
distal: farthest from the center point, in present embodiments it is the farthest from the chest tube
embedded: to set firmly in the container
empyema: the accumulation of pus in a body cavity
established in practice: a medical device settled in a position while performing a procedure
hold out area: a small flap, strap, loop, or similar appendage, used for pulling
in situ: in position, not extending beyond the focus or level of origin
incision: a cut or surgical wound usually made with a knife
intercostal space: between the ribs
intrathoracic: within the cavity of the chest
indicia: a notice of information
outlet nozzle: the distal end of a unidirectional valve
lung resection: the surgical removal of all or part of the lung
manometer: an instrument for indicating the pressure of any fluid or the difference in pressure between two fluids
mid-clavicular line: vertical line passing through the midpoint of the bone connecting the breastbone with the shoulder blade
mounting straps: a flexible material for holding up or securing the container
open cardiac procedures: any type of surgery where the chest is cut open and surgery is performed on the muscles, valves, or arteries of the heart
PAL: prolonged air leak
patient: any mammal
percutaneous: denoting the passage made through unbroken skin and tissue by needle puncture
pleural: The serous membrane enveloping the lungs and lining the walls of the thoracic cavities
pleural effusion: increased fluid in the pleural space
pneumothorax: presence of free air in the pleural or thoracic cavity
Prolonged Air Leak: continued bubbling of air around a chest tube after being in situ for 48 hours
proximal: nearest the point of origin, in present and particular embodiments it is nearest to the chest tube
pulmonary: relating to the lungs
pulmonary resection: a procedure performed to remove part of the lung
pus: a yellowish white liquid matter produced in certain infections
reduction coupler: a device that narrows the gauge
securing grip: a device for fastening things together that will not likely fail or give way
slit interstitial: the size of a narrow, lengthwise, straight incision
stoma: a mouth like opening
thorax: the cavity containing the heart, lungs, etc., in the chest
thoracic: in or near the thorax
thoracic trauma: injury, wound, or shock to the thorax
thoracic procedures: a particular course of action, such as surgery, and the like to the thorax
unidirectional: moving in only one direction
Velcro™: a trademark name for a nylon material with tiny hooks and a complimentary surface that can be pressed together or pulled apart for easy fastening and unfastening venting bag: the body of the container

### Notations

¹ Prolonged Air Leak also known as Persistent Air Leak, Post-operative Air Leak, PAL and the like, will be referred to as Prolonged Air Leak.

² Air in or around the thorax, also known as gas, pneumothorax and the like, may be referred to as air.

³ Journal of Thoracic Disease. March 2014. “The anticipation and management of air leaks and residual spaces post lung resection”. Michael Rolf Mueller and Beatrice A. Marzluf

⁴“Estimating hospital costs attributable to prolonged air leak in pulmonary lobectomy”. Marcelo F. Jimen ez, Nuria Novoa and Jose L. Aranda European Journal of Cardio-thoracic surgery vol 2 27 issue 2.

⁵ Agency for Healthcare Research and QualityAppendix B: “Excess Cost and Length of Stay Associated with Voluntary Event Reports in Hospitals Cost of Poor Quality or Waste in Integrated Delivery System Settings”. Publication # 08-0096-EF. Andrew R. Paradis, MBA, Valerie T. Stewart, Ph.D., K. Bruce Bayley, Ph.D., Allen Brown, Andrew J. Bennett

⁶ The coupler is mentioned for description. It is a conventional or typical feature that is considered non-essential to the understanding of the invention thus embodiment is not limited to said coupler; other connection devices may be employed.

⁷ The securing grip (30) is for example purposes, said securing implement may be used, however another conventional fastener or a plurality of fasteners such as clips, tapes, locking or clamping elements, and such, may be employed. In some environments a luer may be employed. Additionally the securing grip does not have to be a snap fit design. This item is not considered essential to the invention thus embodiment is not limited to said fastener, any other securing method or no securing method may be employed.

⁸ The optimal location for placement of present embodiment is dependent upon clinical need. Examples of some conditions that may require venting are pulmonary resection, open cardiac procedures, disease, thoracic trauma and other thoracic procedures, disease, thoracic trauma and other thoracic procedures. However, conditions are for illustration purposes and present embodiment is not limited to such procedures.

### CITATIONS: CROSS-REFERENCE TO RELATED APPLICATIONS

U.S. Pat. No. 8,181,643 B2 System and method for detection and repair of pulmonary air leaks Joseph S. Friedberg

CN 2601634 Y Chitin shim can be stuck capable of preventing air escape on the edge of lung. Chinese

US20090136589A1 Methods for sealing fluid leaks in lung tissue. Traves Dean Crabtree

U.S. Pat. No. 6,328,689 B1 Lung constriction apparatus and method Gonzalez, Muff, Sirokman

U.S. Pat. No. 8,444,690 B2 Bronchoscopic repair of air leaks in a lung. Hugo Xavier Gonzalez

EP 2544755 A1 Chest drainage systems and methods. James Croteau

U.S. Pat. No. 4,372,336 A Chest drainage unit. William D. Cornell

U.S. Pat. No. 4,738,671 A Chest drainage apparatus with check valve. Donald P. Elliott

WO1999010024A1 Portable suction apparatus. David Waller

U.S. Pat. No. 4,592,741 Medical apparatus designed for the aspiration of pneumothorax Michel J. Vincent Jun. 12, 1984 Jun. 3, 1986

U.S. Pat. No. 6,544,192 Patient monitor and method of using same. Eric W. Starr

U.S. Pat. No. RE35225 Fluid recovery system. Steve A. Herweck

U.S. Pat. No. 4,654,029 Electronic drainage system. Nicholas F. D'Antonio Dec. 13, 1982 Mar. 31, 1987 Howmedica, Inc.

U.S. Pat. No. 5,300,050 Drainage device. Frederick A. Everett Jr. May 7, 1993 Apr. 5, 1994 Deknatel Technology Corporation Drainage device

U.S. Pat. No. 5,989,234 Device and system for draining a body cavity and methods related thereto. Michael A. Valerio Jan. 14, 1997 Nov. 23, 1999 Deknatel Technology Corporation

U.S. Pat. No. 6,338,728 Chest drainage device having multiple operation indicators. Michael A. Valerio May 17, 2000 Jan. 15, 2002 Genzyme Corporation

U.S. Pat. No. 3,683913 Underwater drainage apparatus with air flow meters. Robert Bidwell Oct. 5, 1970 Aug. 15, 1972 Deknatel Inc

U.S. Pat. No. 4,569,674 Continuous vacuum wound drainage system. Earl G. Phillips Aug. 3, 1982 Feb. 11, 1986 Stryker Corporation

CN 202724434 U Single-use chest drainage device portable bag. Chinese

U.S. Pat. No. 4,675,010 Thoracic drainage collection system and method. George G. Siposs

WO2009027665A1 Container. David Hill

U.S. Pat. No. 4,569,674 Continuous vacuum wound drainage system. Earl G. Phillips

WO2007024230A1 Aug. 26, 2005 Mar. 1, 2007 Spheric Products Ltd Chest tube drainage system

EP2189171A1 Nov. 25, 2008 May 26, 2010 Covidien A G Mobile chest drainage unit, thoracic catheter system comprising a mobile chest drainage unit and a thoracic catheter, and manufacturing method

EP0080364A1 Nov. 23, 1982 Jun. 1, 1983 Craig Medical Products Limited Drainage bag and manufacture

U.S. Pat. No. 3,572,340 Jan. 11, 1968 Mar 23, 1971 Kendall & Co Suction drainage device

U.S. Pat. No. 4,675,010 Oct. 4, 1985 Jun. 23, 1987 American Omni Medical, Inc. Thoracic drainage collection system and method

U.S. Pat. No. 5,300,050 May 7, 1993 Apr. 5, 1994 Deknatel Technology Corporation Drainage device

U.S. Pat. No. 4,747,844 May 31, 1988, Donald P. Elliot, Chest Drainage Apparatus

I claim:

1. An air venting container comprising:
a. a container as claimed in claim 1 wherein the container body comprises a substantially closed bag defining an interior space, and
b. a container as claimed in claim 1 wherein an air inlet passage comprises a tubular member, having a first end and a second end, an outer surface and an inner surface, and
c. a container as claimed in claim 1 wherein an air outlet passage of the container comprises at least one aperture extending from the outer surface to the inner surface of the container, and
d. a container as claimed in claim 1 wherein the container comprises a mounting portion, and
e. a container as claimed in claim 1 whereby present embodiment provides a container for the collection and aspiration of air from a body, and
f. a container as claimed in claim 1 substantially as here-before described with reference to FIGS. 1, 2, 3, 4, 5, and 6.

2. An air venting container in accordance with claim 1 and further comprising:
a. a container as claimed in claim 2 wherein the container comprises a valve which allows a forward flow and prevents backflow of air, and
b. a container as claimed in claim 2 wherein the container body is comprises a substantially closed bag defining an interior space, and
c. a container as claimed in claim 2 wherein an air inlet passage comprises a tubular member, having a first end and a second end, an outer surface and an inner surface, and
d. a container as claimed in claim 2 wherein an air outlet passage of the container comprises at least one aperture extending from the outer surface to the inner surface of the container, and
e. a container as claimed in claim 2 wherein the container comprises a mounting portion, and
f. a container as claimed in claim 2 whereby present embodiment provides a container for the collection and aspiration of air from a body, and
g. a container as claimed in claim 2 substantially as here-before described with reference to FIGS. 1, 2, 3, 4, 5, and 6.
